(19) United States
(12) Patent Application Publication
(10) Pub. No.: US 2021/0228504 A1
(43) Pub. Date: Jul. 29, 2021

(54) SURFACTANTS FOR TREATMENT OF CONDITIONS THROUGH TARGETED NECROSIS

(71) Applicant: IMUNEKS FARMA ILAC SAN. VE TIC. A.S., Istanbul (TR)

(72) Inventor: Mehmet Nevzat PISAK, Besiktas/Istanbul (TR)

(73) Assignee: IMUNEKS FARMA ILAC SAN. VE TIC. A.S., Istanbul (TR)

(21) Appl. No.: 16/314,236

(22) PCT Filed: Jun. 2, 2017
(86) PCT No.: PCT/TR2017/050242

§ 371 Date: Dec. 28, 2018

(30) Foreign Application Priority Data
Jun. 28, 2016 (TR) PCT/TR2016/050200

(51) Int. Cl. A61K 31/10

(52) U.S. Cl. CPC A61K 31/10

(57) ABSTRACT

The present invention relates to a surfactant, or a pharmaceutically acceptable salt of a surfactant or a stereoisomer of a surfactant, and a pharmaceutical composition including the surfactant, for the treatment of conditions of the cervix/anogenital region and non-melanoma skin cancers (NMSCs). The composition may comprise an anionic or amphoteric surfactant, or a pharmaceutically acceptable salt or a stereoisomer thereof, wherein the surfactant comprises: a hydrophilic moiety selected from the group consisting of a carbonate, a sulfonate, and a sulfate; and at least one pharmaceutically acceptable excipient. Methods of using the surfactant and composition are also disclosed.

### CROSS-REFERENCE TO RELATED APPLICATIONS

The present application claims priority to PCT application no. PCT/TR2016/050200 filed 28 Jun. 2016.

### TECHNICAL FIELD

The present invention relates to the use of surfactants for the treatment of conditions and diseases that would benefit from the induction of necrosis by the local application of specific amounts of surfactants.

### BACKGROUND

Programmed Cell Death (PCD) plays a fundamental role in animal development and tissue homeostasis. Abnormal regulation of this process is associated with a wide variety of human diseases, including immunological and developmental disorders, neuro-degeneration, and cancer.

There are two primary forms of cell death: apoptosis and necrosis. Apoptosis is a process of programmed cell death that occurs in multicellular organisms. Biochemical events lead to characteristic cell changes (morphology) and death. These changes include blebbing, cell shrinkage, nuclear fragmentation, chromatin condensation, chromosomal DNA fragmentation, and global mRNA decay. Between 50 and 70 billion cells die each day due to apoptosis in the average human adult. For an average child between the ages of 8 and 14, approximately 20 billion to 30 billion cells die a day.

Cell lysis (cytolysis) is the destruction of cells by rupture or disintegration of the membrane and loss of cell contents. Under normal circumstances it is usually induced by viruses, antibodies and/or by a hypotonic environment.

Necrosis on the other hand is a form of cell injury which results in the premature death of cells by lysis. Apoptosis can be beneficial whereas necrosis is harmful when uncontrolled.

Nonmelanoma skin cancer (NMSC) is the most common cancer in Caucasians. Basal cell carcinoma (BCC) accounts for 75% of cases, and is a slow-growing, locally invasive epidermal tumor with a metastatic rate of <0.1%. Squamous cell carcinomas of the skin and their precursors, actinic keratosis as well as basal cell carcinomas are classified as non-melanocytic skin cancer and belong to the group of epithelial skin tumors. This tumor entity is one of the most common forms of malignant cancer in western countries with an incidence of approximately 100-170 per 100,000 inhabitants per year in Europe.

Basal cell carcinoma accounts for more than 90 percent of all skin cancers in the United States and is the most common of all cancers. Typically, it is a slow-growing cancer that is rarely metastatic, it is an epithelial neoplasm that is believed to derive from the basal layer of the epidermis or follicular epithelium. The classic histologic presentation of BCC is that of nodules and/or strands of atypical basaloid cells that show nuclear palisading, cellular apoptosis, and scattered mitotic activity.

Squamous cell carcinoma (SCC) accounts for the majority of the remainder of cases. Squamous cell carcinoma, also known as squamous cell cancer, is one of the main types of skin cancer that begins from squamous cells in the skin.

SCC and BCC are generally treated by surgical excision, Mohs surgery or electrodessication and curettage. Non-surgical options for the treatment of SCC and BCC include topical chemotherapy, topical immune response modifiers, photodynamic therapy (PDT), radiotherapy, and systemic chemotherapy. The use of topical therapy, such as Imiquimod cream and PDT is generally limited to premalignant (i.e., actinic keratoses) and in situ lesions. Radiation therapy is a primary treatment option for patients in whom surgery is not feasible and is an adjuvant therapy for those with metastatic or high-risk cutaneous SCC. At this time, systemic chemotherapy is used exclusively for patients with metastatic disease. All squamous cell carcinoma lesions are thought to begin via the repeated, uncontrolled division of cancer stem cells of epithelial lineage or characteristics. In contrast to BCC, SCC has a significant recognized rate of metastasis (0.3-3.7%), the majority of which occur from within a subgroup of high-risk SCC.

Incidence of NMSC has significantly increased up to 10% per annum, and currently 2-3 million new cases of NMSC are diagnosed worldwide every year. Most countries do not have cancer registries for NMSC and reported figures are likely to be underestimated. Incidence rates of NMSC increase closer to the equator, with the highest reported rates in the northern territories of Australia.

Actinic keratosis is caused by chronic sun damage to the skin and is considered a milder form of NMSC. Actinic keratosis is usually the first lesion in a disease continuum that progresses to invasive SCC. The risk of progression from actinic keratosis to SCC depends on a number of patient factors. The risk of transformation is enhanced in patients with increased solar damage, immunosuppression, and those of advanced age. The presence of actinic keratosis is a risk factor for non melanoma skin cancers and they are precursors of squamous cell carcinoma. The treatment of actinic keratosis is also very important to prevent the development of the disease in to SCC.

Infection, scarring and hemorrage are significant risks involved with curettage, cautery and other surgical options. Although radiotherapy has been efficient in the treatment of NMSC, it has been shown to increase the risk of subsequent BCC and SCC. Toll-like receptor (TLR) agonists, such as imiquimod, are efficient in the treatment of certain types of NMSC but they induce local skin reactions in a large percentage of the patients.

It is known that oncogenic viral strains can develop into cancer. Although there have been many developments in cancer research and specifically in this area, current therapies have a high incidence of side effects, which creates patient compliance issues and decreases the efficacy of these therapies. Cervical intraepithelial neoplasia (CIN), also known as cervical dysplasia, is the potentially premalignant transformation and abnormal growth (dysplasia) of squamous cells on the surface of the cervix. CIN most commonly occurs on the cervix at the squamo-columnar junction, but can also occur in vaginal walls and vulvar epithelium. All these infections are considered pre-cancerous conditions of the cervix/anogenital region. The major cause of CIN is chronic infection of the cervix with the sexually transmitted human papillomavirus (HPV), especially the high-risk HPV types 16-18. Over 200 types of HPV have been identified. About a dozen of these types appear to cause cervical dysplasia and may lead to the development of cancers of the cervix/anogenital region. There are many similarities between adenocarcinoma and SCC of the cervix, and they are treated mostly in the same manner. However, there are also several differences in epidemiology, prognostic factors, patterns of failure after primary treatment, and possibly in response to specific treatments due to the fact that most new treatments are focused on immunomodulation and the responses vary because the lesions cannot be targeted directly.

There have been more developments in agents that exert their therapeutic effects through apoptosis and immunomodulation, but prior research in necrosis inducing or cytolytic agents has met with difficulty due to their cytotoxicity for healthy cells.

### BRIEF DESCRIPTION OF THE FIGURES

FIG. 1 illustrates the effect of an active surfactant mixture on HaCaT cells in accordance with embodiments of the present invention.

FIG. 2 illustrates the effect of an active surfactant mixture on HeLa cells (HPV-18 positive cervical adenocarcinoma cell line) in accordance with embodiments of the present invention.

FIG. 3 shows results from an MTS assay on HeLa cells 24 hours after treatment in accordance with embodiments of the present invention.

FIG. 4 shows results from an MTS assay on SiHa (SCC cell line) cells 24 hours after treatment in accordance with embodiments of the present invention.

FIG. 5 shows results from an MTS assay on HaCat cells 24 hours after treatment in accordance with embodiments of the present invention.

FIGS. 6 and 7 show the effect of tested compounds on HeLa cell death and HaCat cell death, respectively, in accordance with embodiments of the present invention.

### DESCRIPTION OF EMBODIMENTS

The present invention is targeted towards the induction of local targeted necrosis through a cytolytic effect for the treatment of pre-cancerous conditions of the cervix /anogenital region and non-melanoma skin cancers (NMSCs) by the application of surfactants, including application of specific amounts of surfactants in some embodiments. For example, the induction of necrosis of the areas affected by SCC through the local application of surfactants would be beneficial in the treatment of this disease as the surprising effects of the surfactants used on SiHa cells (SCC cell line) demonstrates. Furthermore the same effect also observed on SiHa and HeLa cells (HPV positive cancer cells) also demonstrates the efficacy of the surfactants to help prevent the progression of oncogenic HPV infections to pre-cancerous conditions of the cervix/anogenital region and possibly into cancer.

In one embodiment, a surfactant, or a pharmaceutically acceptable salt of a surfactant or a stereoisomer of a surfactant, is provided for treatment of pre-cancerous conditions of the cervix/anogenital region and non-melanoma skin cancers (NMSCs). Preferably, anionic and/or amphoteric surfactants are used. The use of the anionic surfactant is due to the anionic functional group of the surfactant used in the composition that demonstrated the surprising effects during the in-vitro study detailed below and in the trial detailed in PCT application no. PCT/TR2016/050200. Thus, anionic or amphoteric surfactants would have this specific anionic functional group that was efficient as a therapeutic and they would be a preferred embodiment of this invention based on the aforementioned results.

The above surfactant may have alternative components or elements, which may also be combined in various applicable and functioning combinations within the scope of the present invention.

The surfactant may have at least one of the following formulas (I) to (XXIII):

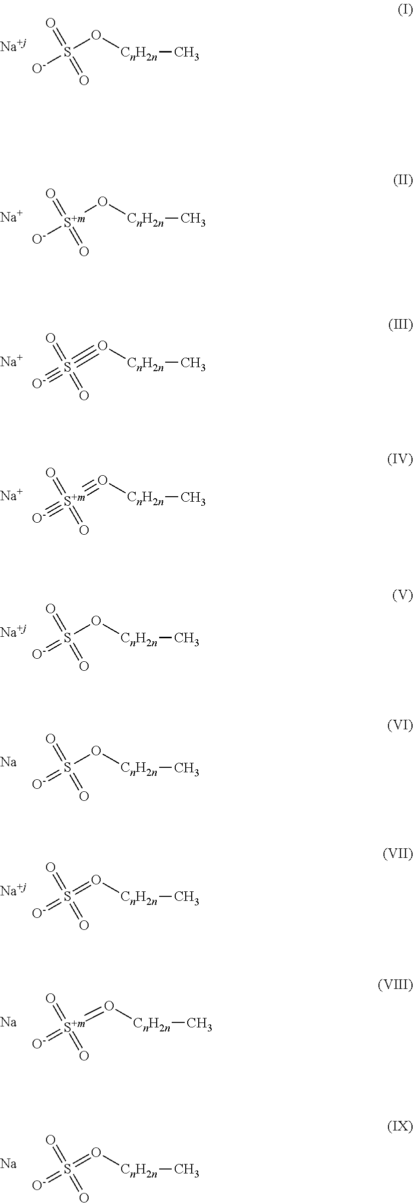

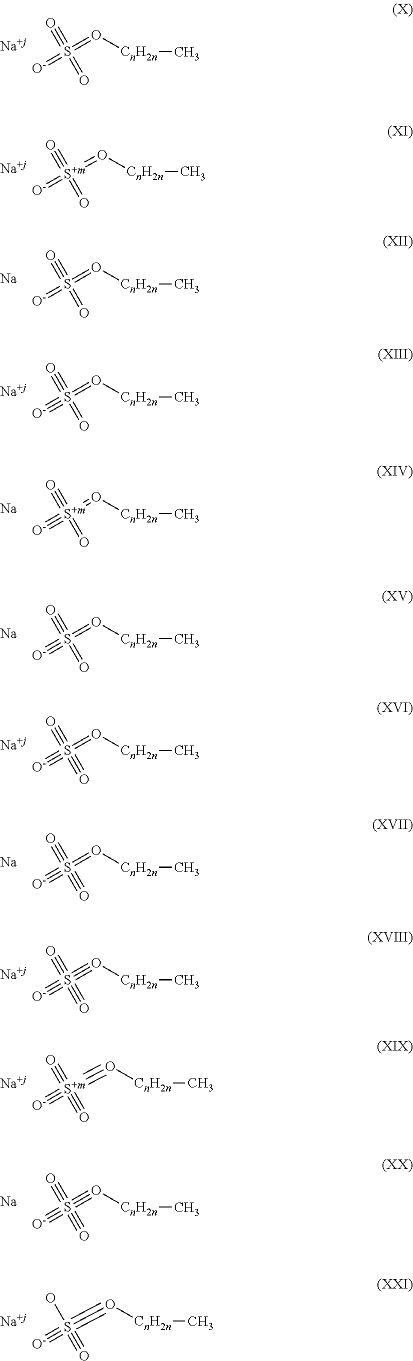

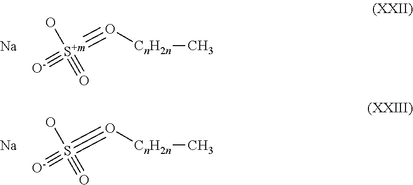

wherein
m is an integer from 1 to 10;
n is an integer from 3 to 31; and
j is an integer from 1 to 10.

In the surfactant formulas above, n may also be an integer from 5 to 21, 7 to 15, or 7 to 25 in other embodiments. The surfactant may be selected from the group consisting of an anionic surfactant, an amphoteric surfactant, and mixtures thereof. The surfactant may further comprise a hydrophilic moiety selected from the group consisting of a carbonate, a sulfonate, and a sulfate also due to the fact that the surprising results in PCT application no. PCT/TR2016/050200 and the study detailed below were achieved with a surfactant that has an anionic functional group, such as an anionic or amphoteric surfactant which had a hydrophilic moiety.

In another embodiment, a composition for treatment of pre-cancerous conditions of the cervix/anogenital region and non-melanoma skin cancers (NMSCs) is provided, the composition comprising: an anionic or amphoteric surfactant, or a pharmaceutically acceptable salt or a stereoisomer thereof, wherein the surfactant further comprises a hydrophilic moiety selected from the group consisting of a carbonate, a sulfonate, and/or a sulfate and at least one pharmaceutically acceptable excipient.

The above composition may have the following alternative components, which may also be combined in various applicable and functioning combinations within the scope of the present invention.

The anionic or amphoteric surfactant of the composition may have one of the formulas (I)-(XXIII) as listed above. The surfactant may be selected from the group consisting of sodium lauryl sulphate, potassium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate their therapeutically active stereoisomers, their pharmaceutically suitable salts, and combinations thereof.

The surfactant may have one of the following weight percentages in the composition: between 0.1% to 20% by weight of the composition; between 0.3% to 10% by weight of the composition; and between 0.3% to 5% by weight of the composition.

The composition may be formulated for topical administration. The composition may be in the form of a gel, a cream, an ointment, a liquid, a suspension, a solution, an emulsion, a foam, a patch, or an aerosol.

The active agents of the present invention exert their therapeutic effect through cytolysis and the consequential local necrosis of an affected cell population. Although the active agent of the invention may include cytolytic properties, it has been surprisingly discovered that it has minimal to no side effects to patients as detailed in PCT application no. PCT/TR2016/050200, to which this application claims priority.

It can be said that conventional chemotherapeutic agents not only elicit apoptosis but other forms of nonapoptotic death such as necrosis, autophagy, mitotic catastrophe, and senescence. Treatments that induce necrotic death in cancer cells include photodynamic treatment (PDT) and alkylating DNA damaging agents. Several other chemicals or drugs, such as β-lapachone, apoptolidin, and honokiol, have been demonstrated to induce cancer cell death through necrosis.

Necrosis of cancer cells engenders cellular debris and many immune-stimulatory components to stimulate immune function, contrary to the immune suppression caused by common chemo agents and making necrosis a better cell death mode.

Induced cytolysis of affected cell populations, such as those in the cervical or anogenital region infected by oncogenic viral strains, would accelerate release of both mature and immature virus particles that will be exposed to the denaturing potency of the anionic surfactant and will also be subsequently inactivated. A similar surprising effect is evidenced by the use of the surfactant on SiHa cells (bearing SCC) where the SCC bearing cell line goes through necrosis due to the cytolytic effect of the surfactant. Furthermore, massive locally induced cytolytic effect and consequential necrosis due to the cytolytic effect would induce secretion of proinflammatory cytokines that will provoke an adaptive immune response which would also limit the recurrence of these conditions.

In accordance with embodiments of the present invention, a local targeted treatment, without the burden of systemic administration and a high level of efficacy in the treatment of pre-cancerous conditions of the cervix/anogenital region and non-melanoma skin cancers (NMSCs), creates a new treatment modality for millions of patients suffering from these potentially lethal conditions.

Although the clinical study detailed in PCT application no. PCT/TR2016/050200 has discovered that certain surfactants were highly efficient for the treatment of conditions caused by the human papilloma virus (HPV), the in-vitro study detailed below has surprisingly shown that surfactants as detailed above can be a highly effective treatment for the case when targeted necrosis is needed through a cytolytic effect, with mild to no side effects especially when the condition to be treated is a condition involving an epithelial tumor such as BCC and/or SCC (including AK which is a precursor to SCC) or when it is a precancerous condition of the cervix/anogenital region, which are mainly caused by oncogenic viral strains, all of which are evidenced by the surprising discoveries of the study detailed below. Due to the nature of the cell lines used in the study which are cancer and HPV positive cell lines and the cytolytic effect of the surfactant inducing necrosis in a dose dependent manner, the present invention provides a completely new treatment modality with minimal side effects through its cytolytic effect and consequential targeted necrosis.

The present invention also has the advantage of its local application which creates a more targeted approach compared with other therapies. This local application creates more exposure of the surfactants to the cancerous or pre-cancerous cells with less side effects. This is in contrast to the prior teachings of the state of the art.

For example, although U.S. Pat. No. 4,822,605 has described a treatment with a non-ionic surfactant, only a combination therapy is disclosed requiring other agents which have serious side effects as a “lymphotoxin, tumor necrosis factor, interleukin, target cell lysis factor and carcino-breaking factor”.

Furthermore, PCT application WO 2014018874 describes a treatment for skin cancer, “comprising the step(s) of: applying an isothiocyanate functional surfactant to an area affected by skin cancer, wherein the isothiocyanate functional surfactant comprises at least one isothiocyanate functional group associated with an aliphatic and/or aromatic carbon atom of the isothiocyanate functional surfactant.” Although WO 2014018874 teaches that an isothiocyanate functional surfactant can be used for the treatment of skin cancer, it also teaches that the isothiocyanate functional surfactant should be removed. The patent does not specify the reason why, but the state of the art and our in-vitro study have shown that the removal may have been due to the cytotoxicity of such compounds. In contrast, the surfactants disclosed in the present invention have shown more affinity to cancer cells than healthy cells; an augmented cytolytic effect towards HeLa cells rather than the HaCat cells, which explains the exceptional safety profile.

Contrary to the state of the art explained above, the present invention does not require a wash off or removal of the applied surfactant, as it has been shown to have an excellent safety profile in PCT application no. PCT/TR2016/050200 as a leave on topical product, even when it was applied daily on the sensitive area of the genital mucosa for more than 8 weeks. Accordingly, the surfactant active ingredients and/or compositions of the present invention may be left on the skin after application and does not require an active removal step. Furthermore the surfactant active ingredients and/or compositions of the present invention have the advantage of daily application due to its excellent safety profile as evidenced by the study detailed in PCT/TR2016/050200. The completion of the study has also surprisingly proven that the treatment with the surfactant composition of the present invention is a better treatment at a shorter treatment period compared to other products on the market, such as aldara (imiquimod 5%) and even compared to imiquimod 3.75% which still has to be removed after 8 hours, whereas the composition of the present invention with a median treatment period of less than 4 weeks does not have to be removed or washed off.

The surprising effect shown and detailed in the in-vitro study below coupled with the surprising effects of the same compound(s) shown in PCT application no. PCT/TR2016/050200 clearly demonstrate that surfactants and/or compositions of the present invention, and in particular anionic and amphoteric surfactants, have a therapeutic effect for the treatment of diseases which would benefit from the induction of localised targeted necrosis without the usual associated side effects of current therapies in use and in the prior state of the art.

Having demonstrated the excellent safety profile of the surfactants of the present invention in PCT application no. PCT/TR2016/050200, and in combination with the surprising results of the in-vitro study detailed below, it is clearly shown that the present invention is an ideal therapeutic for pre-cancerous conditions of the cervix /anogenital region and non-melanoma skin cancers (NMSCs). Furthermore, the in-vitro study below has also shown that the active mixtures containing the surfactants of the present invention have more affinity for the cancer and HPV positive HeLa cells, due to the fact that the cytolytic effect was observed earlier in the HPV-18 positive cervical adenocarcinoma cell line (HeLa cells) than in healthy keratinocytes, which helps to explain the excellent safety profile of the present invention, although the present invention is not limited to such a theory in any case.

In-Vitro Study Methodology

Cells were seeded the day before testing in 12-well plate dishes in complete medium. Testing was performed using the following serial dilutions of active mixture-AM (surfactant; propylene glycol; carboxypolymethylene) and corresponding dilutions of a placebo (propylene glycol; carboxypolymethylene) as a control:
1. 1× (˜0.8 wt % surfactant), undiluted AM
2. 2× (0.4 wt % surfactant)
3. 4× (0.2 wt % surfactant)
4. 8× (0.1 wt % surfactant)
5. 10× (0.08 wt % surfactant)
6. 20× (0.04 wt % surfactant)
7. 50× (0.016 wt % surfactant)
8. 100× (0.008 wt % surfactant)
9. 200× (0.004 wt % surfactant)

Cells were observed at various time points (immediately upon applying of tested drug, upon 5 min, 1 h and 24 h). High cytolytic effect was observed starting from undiluted substance up to a 20× dilution (lead to complete lysis of the cells immediately after addition or within first hour). Thereafter, decrease in the cytolytic effect was detected. However, it seems that decrease in cytolysis was not completely gradual; at some point the substance was not cytolytic to the cells anymore (around 100× dilution). On the other hand, the placebo did not show a cytotolytic effect in any tested concentration. The overall effect on cell's morphology and survival was similar between healthy keratinocytes (HaCaT), or HPV-18 positive cervical adenocarcinoma cell line (e.g. HeLa). However, in HeLa cells cytolytic effect was observed earlier then in healthy keratinocytes (complete cell loss within first hour vs. 24 hours in the case of HaCaT cells).

During the treatment of HaCat cells with active mixture, cell-cell interactions were lost, membrane integrity was compromised and cell lysis was observed. After 24 hours of treatment, complete lysis was observed. In parallel, the same dilution of a placebo did not result in cell damage.

For HeLa cells, complete cell loss was evident at an earlier time point, compared to HaCaT cells (within first hour) while placebo remained ineffective. Advantageously, the composition of the present invention with an anionic surfactant has more affinity towards HPV bearing oncogenic cells (HeLa cells) rather than healthy keratinocytes (HaCaT cells) which demonstrates why the composition has a very good safety profile although it is cytolytic and induces necrosis.

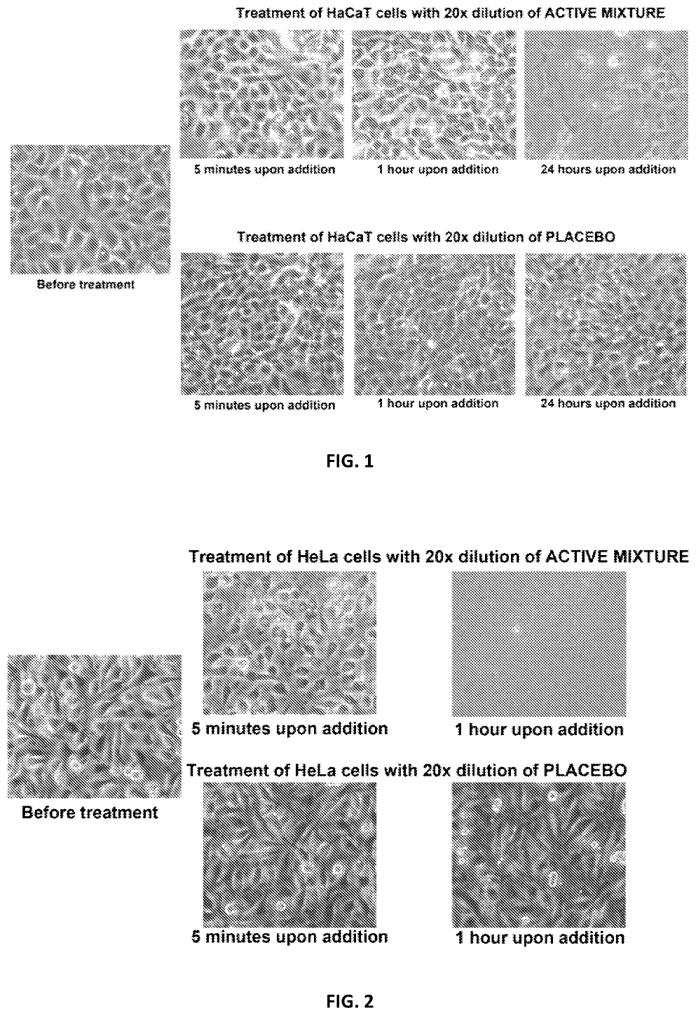

The effect of the tested drug at various concentrations on cell morphology and survival is summarized in Table 1 below, and some representative image captures are presented in FIGS. 1 and 2. FIG. 1 illustrates the effect of an active mixture (20× dilution, ˜0.04 wt % surfactant) and a placebo on HaCaT cells after 5 minutes, 1 hour, and 24 hours. FIG. 2 illustrates the effect of an active mixture (20× dilution, 0.04 wt % surfactant) and a placebo on HeLa cells after 5 minutes and 1 hour. As can be seen, cell death occurs over time with the addition of the active mixtures, and at a much higher rate of cell death with the active mixtures as compared to the pacebo.

TABLE 1
The effect of various concentrations of active
mixture on HaCat cell's morphology and survival

| Dilution | Duration of treatment | | | |
|---|---|---|---|---|
| of active | 0 minutes | 5 minutes | 1 hour | 24 hours |
| mixture | Overall effect on HaCaT cell's morphology and/or survival | | | |
| 1x | Complete | | | |
| | cell death | | | |
| 2x | Complete | | | |
| | cell death | | | |
| 4x | Visible lysis, | Complete | | |
| | empty cells | cell death | | |
| 8x | Lost of | Visible | Complete | |
| | cell-cell | lysis | cell death | |
| | contact, | | | |
| | membrane | | | |
| | integrity is | | | |
| | compromised | | | |
| 10x | Lost of | Visible | Complete | |
| | cell-cell | lysis | cell death | |
| | contact, | | | |
| | membrane | | | |
| | integrity is | | | |
| | compromised | | | |
| 20x | Majority of | Lost of | Cell | Complete |
| | cells look | cell-cell | leakage | cell death |
| | viable | contact, | is visible, | |
| | | membrane | cells | |
| | | integrity is | appear | |
| | | compromised | empty | |
| | | in many cells | | |
| 50x | Majority of | Majority of | Some | Cell leakage |
| | cells look | cells look | cell-cell | is visible, |
| | viable | viable | contacts are | some cells |
| | | | lost, many | are still |
| | | | viable cells | unharmed |
| | | | remained | |
| 100x | Viable | Viable | Viable | Majority of |
| | cells | cells | cells | viable cells |
| 200x | Viable | Viable | Viable | Majority of |
| | cells | cells | cells | viable cells |

MTS Assays (Cell Viability Test)

Since dilutions ranging from 1× to 20× had a high cytolytic effect, for cell viability assay (MTS assay), the following dilutions of active mixture were applied:
1. 50× (0.016 wt % surfactant)
2. 75× (0.012 wt % surfactant)
3. 100× (0.008 wt % surfactant)
4. 150× (0.006 wt % surfactant)
5. 200× (0.004 wt % surfactant)

Cells were seeded in 96 well plates the night before treatment and treated with various dilutions of active mixture or placebo for 24 hours. The effect of these treatments was monitored on cell's viability using MTS Cell Proliferation Assay (Promega CellTiter 96® AQueous One Solution Cell Proliferation Assay). Obtained results are presented in FIGS. 3, 4 and 5.

Referring to FIG. 3, results from an MTS assay on HeLa cells 24 hours after treatment are shown. A: Active mixture; P: Placebo. Relative cell's viability of treated cells was calculated as a percentage of untreated cells viability, which was set as 100%. Data are presented as the mean±standard deviation of at least three independent experiments performed in 6-plicates for each concentration. It was found that the half maximal inhibitory concentration (IC₅₀) for HeLa cells is approximately 90× dilution of active mixture. No effect on cells' viability was observed for the placebo, which shows the surfactant as an active component of the drug.

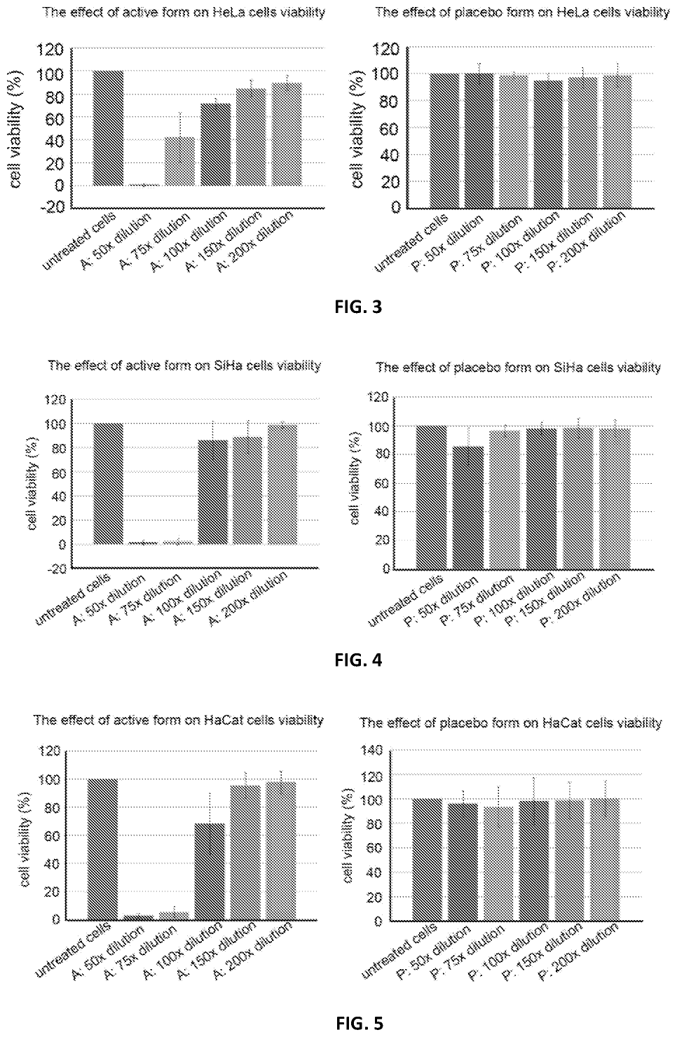

Referring to FIG. 4, results from an MTS assay on SiHa cells 24 hours after treatment are shown. A: Active mixture; P: Placebo. Relative cell's viability of treated cells was calculated as a percentage of untreated cells viability, which was set as 100%. Data are presented as the mean±standard deviation of at least three independent experiments performed in 6-plicates for each concentration. It was found that the half maximal inhibitory concentration (IC₅₀) for SiHa cells (HPV 16 positive, squamous cell carcinoma) was difficult to determine because there is a sharp transition between the effect of 75× dilution (almost complete cytolytic effect upon 24 hours) and 100× dilution (more than 80% of cells survived). This “all or nothing” effect has been observed in all analyzed cell lines (perhaps in a milder form for HeLa cells). As shown for HeLa cells, the placebo composition did not exert any cytotolytic effect.

Referring to FIG. 5, results from an MTS assay on HaCat cells 24 hours after treatment are shown. A: Active mixture; P: Placebo. Relative cell's viability of treated cells was calculated as a percentage of untreated cells viability that was set as 100%. Data are presented as the mean ±standard deviation of at least three independent experiments performed in 6-plicates for each concentration. It has been found that the half maximal inhibitory concentration (IC₅₀) for HaCat cells is approximately between 80-90× dilution (although “all or nothing” effect was obvious in this cell line as well). No effect on cells'viability was observed for the placebo composition, confirming that the surfactant is an active component of the analyzed mixture.

Annexine V/Propidium Iodide Assay

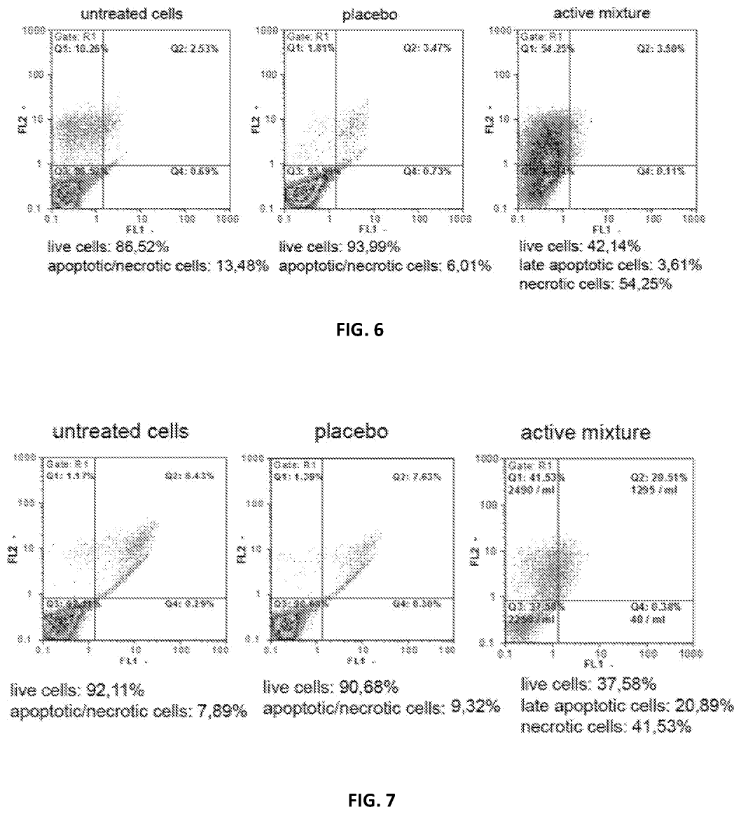

In order to test the effect of surfactants on induction on cell death (apoptosis/necrosis processes) an Annexine V/Propidium Iodide assay was used. Preliminary results on HeLa and HaCat cells (n=1) suggest that the mechanism of cell death is necrosis which is detected by measuring the intensity of red florescent cells stained with Propidium Iodide (PI) by Flow cytometry. By treatment of HeLa cells with 80× dilution of active form of the drug, the integrity of the plasma and nuclear membranes were disturbed, which allowed PI to pass through the membranes and stain the cells. The effect of tested compounds on HeLa cell death and HaCat cell death are illustrated in FIGS. 6 and 7. Preliminary results of the applied apoptosis/necrosis test indicate that the mechanism of cell death upon treatment with an active mixture is cell necrosis. Placebo treatment did not induce the cell death.

The active mixture including the surfactants of the present invention can be used for the treatment of non-melanoma skin cancers, conditions caused by oncogenic strains of HPV such as cervical dysplasia, and the like when the lesions/affected area can be locally accessed without any surgical intervention and the composition of the present invention can be locally administered.

The concentration for the local application of a surfactant composition of the present invention includes 0.2 wt % to 35 wt % of a surfactant. The surfactant is preferably an anionic or an amphoteric surfactant in one example.

Because vaccines would only have effects towards certain oncogenic strains, these would not create a universal prophylaxis for cervical dysplasia or other conditions caused by pre-cancerous cells in the cervical/anogenital region. Advantageously, due to the specific mechanism of action of the treatment of the present invention through targeted necrosis induced through cytolysis, the treatment would not have the limitations and side effects associated with current therapies, systemic administration and will not be only effective against specific oncogenic strains even compared with the most advanced form of vaccines like Gardasil9.

Certain oncogenic HPV strains will not manifest on the skin of the patient as warts but nonetheless can be cancer causing strains (HPV sub types). These strains can be easily diagnosed by a pap smear, even if they are not diagnosable through a visual inspection. In such cases, the administration of a surfactant or surfactants of the present invention would still be an effective treatment in order to prevent these oncogenic strains from turning into cervical dysplasia and furthermore into cervical cancer.

The foregoing embodiments and examples have been provided merely for the purpose of explanation and are in no way to be construed as limiting of the present invention. While the present invention has been disclosed with reference to embodiments, the words used herein are intended to be words of description and illustration, rather than words of limitation. While the present invention has been described with reference to particular materials and embodiments, the present invention is not intended to be limited to the particulars disclosed herein. Rather, the present invention extends to all functionally equivalent structures, materials, and uses, such as are within the scope of the appended claims. Changes may be made, within the purview of the appended claims, as presently stated and as may be amended, without departing from the scope and spirit of the present invention. All terms used in this disclosure should be interpreted in the broadest possible manner consistent with the context.

1-21. (canceled)

22. An anionic or amphoteric surfactant for use in the treatment of pre-cancerous conditions of the cervix/anogenital region and non-melanoma skin cancers (NMSCs).

23. The surfactant for use according to claim 22, wherein the surfactant further comprises a hydrophilic moiety selected from the group consisting of a carbonate, a sulfonate, and a sulfate.

24. The surfactant for use according to claim 22, wherein the surfactant is selected from the group consisting of sodium lauryl sulphate, potassium lauryl sulfate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate and their salts and/or stereoisomers, and a combination thereof.

25. The surfactant for use according to claim 22, wherein the surfactant is administered onto a skin area 1 to 3 times daily by the application of the surfactant onto the affected area.

26. The surfactant for use according to claim 22, wherein the pre-cancerous condition of the cervix/anogenital region is cervical intraepithelial neoplasia.

27. The surfactant for use according to claim 22, wherein the non-melanoma skin cancer is basal cell carcinoma, squamous cell carcinoma or actinic keratosis.

28. The surfactant for use according to claim 22, wherein the pre-cancerous condition of the cervix/anogenital region is caused by HPV.

29. A pharmaceutical composition for use in the treatment of pre-cancerous conditions of the cervix/anogenital region and non-melanoma skin cancers (NMSCs), the composition comprising: an anionic or amphoteric surfactant, or a pharmaceutically acceptable salt or a stereoisomer thereof, wherein the surfactant further comprises a hydrophilic moiety selected from the group consisting of a carbonate, a sulfonate, and/or a sulfate and at least one pharmaceutically acceptable excipient.

30. The pharmaceutical composition for use according to claim 29, wherein the surfactant is selected from the group consisting of sodium lauryl sulphate, potassium lauryl sulfate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate and their salts and/or stereoisomers, and a combination thereof.

31. The pharmaceutical composition for use according to claim 29, wherein the surfactant is between 0.2% to 35% by weight of the composition.

32. The pharmaceutical composition for use according to claim 31, wherein the surfactant is between 0.3% to 10% by weight of the composition.

33. The pharmaceutical composition for use according to claim 32, wherein the surfactant is between 0.3% to 5% by weight of the composition.

34. The pharmaceutical composition for use according to claim 29, wherein the composition is formulated for local administration.

35. The pharmaceutical composition for use according to claim 29, wherein the composition is in the form of a gel, a cream, an ointment, a liquid, a suspension, a solution, an emulsion, a foam, a patch, or an aerosol.

36. The pharmaceutical composition for use according to claim 29, wherein the pre-cancerous condition of the cervix/anogenital region is cervical intraepithelial neoplasia.

37. The pharmaceutical composition for use according to claim 29, wherein the non-melanoma skin cancer is basal cell carcinoma, squamous cell carcinoma or actinic keratosis.

38. The pharmaceutical composition for use according to claim 29, wherein the pre-cancerous condition of the cervix/anogenital region is caused by HPV.

39. The surfactant for use according to claim 23, wherein the surfactant is administered onto a skin area 1 to 3 times daily by the application of the surfactant onto the affected area.

40. The surfactant for use according to claim 24, wherein the surfactant is administered onto a skin area 1 to 3 times daily by the application of the surfactant onto the affected area.

41. The surfactant for use according to claim 23, wherein the pre-cancerous condition of the cervix/anogenital region is cervical intraepithelial neoplasia.
